(19) United States
(12) Patent Application Publication
(10) Pub. No.: US 2023/0326608 A1
(43) Pub. Date: Oct. 12, 2023

(54) METHODS AND SYSTEMS FOR CLASSIFYING A MALIGNANCY RISK OF A KIDNEY AND TRAINING THEREOF

(71) Applicant: Siemens Healthcare GmbH, Erlangen (DE)

(72) Inventors: Sasa Grbic, Plainsboro, NJ (US); Bernhard Geiger, Cranbury, NJ (US); Philipp Hölzer, Bubenreuth (DE)

(21) Appl. No.: 18/183,973

(22) Filed: Mar. 15, 2023

(30) Foreign Application Priority Data
Apr. 7, 2022 (EP) 22167134.0

(51) Int. Cl. G16H 50/30; G16H 50/20

(52) U.S. Cl. CPC G16H 50/30; G16H 50/20

(57) ABSTRACT

A computer-implemented method is provided for classifying a malignancy risk of a kidney, in particular a human kidney. Imaging data of an anatomy of a subject patient at least partially includes a representation of a kidney of the subject patient. A first neural network segments at least one region of the kidney representation based on the imaging data. A second neural network detects one or more suspected lesions of the segmented kidney representation. A third neural network classifies the detected suspected lesion with a malignancy risk. The third neural network is a deep profiler.

### RELATED APPLICATION

This application claims the benefit of EP 22167134.0, filed Apr. 7, 2022, which is hereby incorporated by reference in its entirety.

### TECHNICAL FIELD

Examples generally relate to methods and systems for classifying a malignancy risk of a kidney. Further examples relate to methods and systems for training a machine learning algorithm to classify a malignancy risk of a kidney.

### BACKGROUND

Determining the malignancy risk for renal lesions is essential for patient management. Especially, the distinction of cysts, which are present in more than 50% of people above 50 years, and cancerous lesions is important. Imaging, especially computed tomography (CT) and magnetic resonance imaging (MRI), plays a crucial part in determining the malignancy risk for complex lesions. However, often the criteria defined are ambiguous and not consistent between physicians. This leads to suboptimal outcomes for patients.

For some of the lesions, ultrasound imaging can be used to distinguish cysts. However, this approach only works for “simple cysts,” which only have clear fluid inside and are harmless. More complex cysts need to be examined with more advanced imaging, such as CT and MRI.

There are several guidelines to examine and report suspected lesions, such as the Bosniak criteria. For example, the Bosniak criteria defines a cystic renal mass as a mass that, based on subjective visual inspection, is composed of less than approximately 25% enhancing components. Therefore, masses with approximately 25% or more enhancing components are considered solid and not classifiable by the Bosniak classification.

The distinction between cystic and solid renal masses is important. Solid masses behave more aggressively and have a higher propensity for local recurrence and metastatic disease than cystic masses. Therefore, their management and prognosis differ. As a corollary, it is important not to mistake a necrotic RCC for a cystic renal cell carcinoma (and vice versa), as the former is aggressive, and the latter is typically indolent.

However, the guidelines are not very specific, which can lead to large user variability and disagreements between individual readers. Thus, the risk assessment and ultimately the patient management can be quite different depending on who is examining the imaging data. Additionally, as the guidelines are based on “simple” appearances that can be interpreted by physicians, the performance of such a classification is limited as much more sophisticated imaging features are not incorporated. This sometimes leads to sub-optimal patient outcomes.

### SUMMARY AND DESCRIPTION

Thus, the classification of malignancy risks of a kidney, in particular a human kidney, may be improved.

A first aspect refers to a computer-implemented method for classifying a malignancy risk of a kidney, in particular a human kidney. The method therefore includes the acts of providing imaging data of an anatomy of a subject patient, wherein the imaging data includes at least partially a representation of a kidney of the subject patient, using a first neural network to segment at least one region of the kidney representation which is based on the imaging data and using a second neural network to detect one or more suspected lesions of the segmented kidney representation. Furthermore, the act of classifying the detected suspected lesion with a malignancy risk using a third neural network is performed, wherein the third neural network is a deep profiler.

By using a deep profiler to determine the malignancy risk of suspected renal lesions, the method can extract complex features from imaging data to create a more accurate risk score. In particular, classifying the malignancy risk improves the patient management for patients with suspected renal lesions.

A second aspect refers to a system for classifying a malignancy risk scoring of a kidney, in particular a human kidney. The system includes an interface configured to provide imaging data of an anatomy of a subject patient, wherein the imaging data includes at least partially a representation of a kidney of the subject patient. Furthermore, the system includes at least a first analyzing unit (processor) configured to use a first neural network to segment at least one region of the kidney representation which is based on the imaging data, a second analyzing unit (processor) which is configured to use a second neural network to detect one or more suspected lesions of the segmented kidney representation, and a deep profiler which is configured to classify the detected suspected lesion with a malignancy risk.

Deploying a system according the second aspect may provide efficient and accurate assessment of the malignancy risk. Based on that, such a system can be deployed anywhere and thus the system deployment is not limited to any certain geographical areas where a specific kidney expert is practicing. In addition, this system can be scaled easily, which means that it may not be limited to a low number of classifications per day as it may be the case when kidney imaging data are classified manually.

In one example according the first and/or second aspect, the third neural network is configured to classify the malignancy risk based on imaging data and non-imaging data, wherein the non-imaging data includes at least histopathologic data. Histopathologic data can pertain to a result of a histopathologic assessment; a microscopic image of a tissue sample can be inspected for tumor-infested tissue, e.g., using fluorescence microscopy.

Non-imaging data may additionally support the classification of malignancy risks since imaging data itself may be incomplete or biased by not accurate enough image recording. In particular, histopathologic data can improve the malignancy risk classification to the extent that false-positive samples can be avoided up to 90%, preferably up to 95% and more preferably up to 99%. Thus, an unnecessary surgical intervention can be avoided for a subject patient. This may reduce healthcare costs and improved patient satisfaction due to reduction in follow-up exams. Furthermore, this may reduce the number of missed malignant lesions and thus may improve the patient survival probability.

In one example according the first and/or second aspect, the deep profiler includes an encoder for extracting imaging features. Utilizing an encoder may support the deep profiler to build at least one task-specific fingerprint. An encoder can contract spatial features in one or more acts to determine a latent feature vector.

In one example according the first and/or second aspect, the encoder is designed as convolutional neural network (CNN), in particular a three-dimensional CNN. Applying a CNN may reduce or even minimize the cost of computing since CNNs make use of local spatial coherence that provides a same weight to some of the edges. This is in particular useful when GPU resources are limited, which may be often the case in medical environments where standard computers or only handheld devices like tablet PCs or smartphones are used for classification. In addition, CNNs may reduce the size of memory needed for execution due to a reduced number of parameters. Compared to a simple deep neural network, a reduced number of hidden layers and nodes may reduce the allocated memory needed for execution.

Moreover, deploying CNNs may help the method or system for classifying a malignancy risk since minor or major changes in the output data may be identified more accurately and retain the reliability of the model. This is based on the fact that convolutions are equivariant to many data transformation operations, which may help to identify how a particular change in input will affect the output. Three-dimensional CNNs may benefit even more from the above identified advantages.

In one example, the deep profiler includes a decoder for estimating at least one malignancy risk indicator. A decoder may expand spatial features in one or more acts, based on a latent feature vector. By estimating one or more approximated imaging data, the decoder can generate synthetic imaging data that can be compared to real-world imaging data again. Any further information like deviations from real-world imaging data and/or deviations from ideal imaging data can be used for further classifications again.

In one example according the first and/or second aspect, the deep profiler includes a task-specific network for generating at least one image signature for classifying at least one malignancy risk. Using one or more image signatures may decrease the false-positive rate of detected lesions of kidneys. Furthermore, comparing imaging data of subject patients with image signatures may be more efficient since specific parameters, which may be characterizing for a high or low malignancy risk, can be emphasized in image signatures.

In one example according the first aspect, the method includes the additional method act: using a fourth neural network to detect anatomical landmarks based on the provided imaging data. This additional act may support the classification since the provided imaging data may be pre-filtered. Thus, not-relevant parts of the anatomy of a subject patient, which are incorporated in the imaging data, can be reduced or even minimized.

In one example according the first and/or second aspect, the fourth neural network is designed as convolutional neural network for image feature extraction. CNNs for image feature extraction may be more beneficial compared to other machine learning algorithms since CNNs are more independent to geometrical transformations like scaling or rotations. Thus, the image feature extraction may be improved.

In one example according the first and/or second aspect, the fourth neural network additionally uses at least one universal non-linear function approximator. Compared to mainly linear function approximators, non-linear function approximators are beneficial due to the fact that they may decrease the error rate compared to the number of parameters in the approximant. In addition, the number of parameters usually correlates with computational efforts. Thus, non-linear functions may help to reduce computational efforts, too. It is shown that in many settings the rate of nonlinear approximation can be characterized by certain smoothness conditions which are significantly weaker than required in the linear theory.

In one example according the first and/or second aspect, the first neural network is designed as convolutional encoder-decoder architecture. This architecture may reduce or even skip connections. Furthermore, a convolutional encoder-decoder setup may support a deep supervision scheme within a framework to improve its structure design compared to a U-Net or deep supervised networks. This is in particular beneficial for 3D volumetric datasets.

In one example according the first and/or second aspect, the first neural network is designed as multi-level feature concatenation and deep supervision architecture. Therefore, bridges may be built directly from the encoder layers to the decoder layers. These bridges may pass information from the encoder forward and then concatenate it with decoder feature layers. Based on that, the first neural network may benefit from local and global contextual information and may achieve improved boundary detection and segmentation results.

In one example, the act of detecting one or more suspected lesions is based on a fully convolutional one-stage object detection (FCOS). The FCOS approach may be anchor box free and proposal free. Additionally, by eliminating the pre-defined set of anchor boxes, FCOS may avoid complicated computation related to anchor boxes such as calculating overlapping. Furthermore, several or even all hyper-parameters related to anchor boxes may be avoided, which may be sensitive to a specific detection performance. Moreover, FCOS may surpass previous one-stage detectors with the advantage of being much simpler and more flexible regarding its detection framework, which may result in an improved detection accuracy.

In one example according the first and/or second aspect, the provided imaging data is based on computer tomography (CT) and/or magnet resonance imaging (MRI). Since image capturing based on CT or MRI is widely applied around the world, a minimum level of quality and accuracy may be guaranteed as inputted imaging data. Furthermore, CT and/or MRI imaging data may be captured in a three-dimensional manner, thus additionally improving and enriching the available imaging data. Since CT and MRI are standard image capturing methods, it may be guaranteed or typical that subject patient can be examined with at least one of CT and/or MRI.

In one example according the first and/or second aspect, the imaging data and/or non-imaging data includes context data relating to data recording information. It may be necessary to access data recording information additionally when, e.g., chromaticity parameters of the imaging data differ from ideal data or data used for training the method or system. Moreover, imaging data may differ based on the image capturing devices from different manufacturers, and thus, it may be necessary to align the different captured imaging data accordingly.

In one example according the first and/or second aspect, the context data includes at least one out of following: data recording device information, contrast configuration information, brightness configuration information, recording direction information, total recording time information, projection type information (e.g., CT: average intensity projection (AIP), maximum intensity projection (MIP) or Minimum intensity projection MinIP)), date and/or time of recording information. Thus, any deviations from other imaging data can be standardized and thus corrected based on the context data.

In one example according the first and/or second aspect, the non-imaging data of the subject patient includes at least one out of the following: age, weight, size, health condition information, gender, nutritional practice. Based on these data which refer to the subject patient any deviations from at least two different imaging data can be at least partly aligned or corrected to improve the classification additionally.

In one example according the first and/or second aspect, the provided imaging data includes at least partially a 3D illustration of the anatomy of the subject patient. This may improve the classification of a malignancy risk since different layers of a detected kidney lesion can be analyzed individually and thus the scoring may be more accurate.

In one example according the first aspect, the method includes the additional act: converting the imaging data from at least a partially 3D illustration of the anatomy of the subject patient to a 2D illustration of the anatomy of the subject patient. This act may be necessary whenever limited computational resources are available. When converting the 3D illustration to a 2D illustration, the volume of imaging data can be reduced and thus, less memory space and computational effort for analyzing the data is needed.

A third aspect refers to a computer-implemented method for training a machine learning algorithm to classify a malignancy risk of a kidney, in particular a human kidney. The method includes the acts of training a first neural network with first training data including imaging data of an anatomy of at least one subject patient, wherein the imaging data includes at least partially a representation of one or more kidneys, training a second neural network with second training data including one or more detected lesions of one or more segmented kidney representations and training a third neural network with third training data of one or more lesions classified with a malignancy risk.

A fourth aspect refers to a system for training a machine learning algorithm to classify a malignancy risk of a kidney, in particular a human kidney, including a first analysis unit configured to train a first neural network with first training data including imaging data of an anatomy of at least one subject patient, wherein the imaging data includes at least partially a representation of one or more kidneys, a second analysis unit configured to train a second neural network with second training data including one or more detected lesions of one or more segmented kidney representations; and a third analysis unit configured to train a third neural network with third training data of one or more lesions classified with a malignancy risk.

A method or system for training a machine learning algorithm to classify a malignancy risk of a kidney may be beneficial due to the fact that it may reduce the error-rate in patient-management of patients which are suspected with renal lesions. In particular, it may increase inconsistency of documented exam information and may decrease the likelihood of user/institution bias. The reason therefore may be seen that a machine learning algorithm may be much more accurate compared to a manual training of a single expert or a group of experts. In particular, inexperienced experts like radiographers may not have the experience compared to a trained machine learning algorithm. Furthermore, by training a machine learning algorithm the classification algorithm may be much faster educated compared to a human expert. In addition, it is nearly impossible for a human expert to analyze and learn his/her manual classification accuracy with such a high number of classified examples compared to a computer-based training.

In one example according to the third and/or fourth aspect, the third training data are based on imaging data and non-imaging data, wherein the non-imaging data includes at least histopathologic data.

Based on histopathologic data, specific information inherent for any malignancy risk classification of kidneys can be added to the machine learning algorithm, which may result in much higher accurate classifications.

In one example according to the third and/or fourth aspect, at least one ground-truth label of the deep profiler is determined based on histopathologic data. Thus, the method or system for training may be more precisely if at least one ground-truth label refers at least partly to histopathologic data.

In one example according to the third aspect, the method includes the following additional act of training a fourth neural network with fourth training data using deep reinforcement learning based on training data related to detected anatomical landmarks, in particular landmarks relating to one or more kidney representations. Based on this, the first, second and/or third training data can be improved with more accurate and relevant data. In particular, irrelevant imaging data information can be reduced or even removed. Thus, the quality of the training data, in particular imaging data, may be improved.

In one example according to the third and/or fourth aspect, an adversarial network is utilized for training of the first neural network to discriminate the output from ground truth. Therefore, the first neural network pushes its output towards the distribution of ground truth, so that it may enhance its performance by refining its output. By doing so, a higher computing efficiency may be achieved since the discrimination does not need to be executed at any inference.

In one example according to the third and/or fourth aspect, at least two different training data fragments are based on the same subject patient. In one example according to the third and/or fourth aspect, the two different training data fragments are indicated as training data of the same subject patient. In one example according to the third and/or fourth aspect, the non-imaging data include at least one indicator configured to serve for patient follow-up diagnosis. Thus, it can be achieved that the training method or system is able to track the malignancy risk of a specific subject patient, in particular over time.

A fifth aspect relates to a computer program including instructions which, when the program is executed by a computer, cause the computer to carry out at least one of the examples of the method according the first aspect or according the third aspect of the invention.

A sixth aspect refers to a non-transitory machine-readable medium having stored thereon a set of instructions, which if performed by one or more processors, cause the one or more processors to carry out at least one of the examples of the method according the first aspect or according the third aspect.

### BRIEF DESCRIPTION OF THE DRAWINGS

Further details and advantages can be taken from the following description of preferred examples in conjunction with the drawings, in which:

FIG. 1 is a schematic drawing depicting a computer-implemented method for classifying a malignancy risk of a kidney according to one implementation;

FIG. 2 is a schematic drawing depicting a system for classifying a malignancy risk scoring of a kidney according to one implementation;

FIG. 3 is a schematic drawing depicting an example implementation of a computer-implemented method for training a machine learning algorithm to classify a malignancy risk of a kidney; and

FIG. 4 is a schematic drawing depicting an example system for training a machine learning algorithm to classify a malignancy risk of a kidney.

### DETAILED DESCRIPTION OF THE EXAMPLE EXAMPLES

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various examples will now be described more fully with reference to the accompanying drawings in which only some examples are shown. Specific structural and functional details disclosed herein are merely representative for purposes of describing examples. Examples, however, may be embodied in various different forms, and should not be construed as being limited to only the illustrated examples. Rather, the illustrated examples are provided as examples so that this disclosure will be thorough and complete and will fully convey the concepts of this disclosure to those skilled in the art. Accordingly, known processes, elements, and techniques, may not be described with respect to some examples. Unless otherwise noted, like reference characters denote like elements throughout the attached drawings and written description, and thus descriptions will not be repeated. The present invention, however, may be embodied in many alternate forms and should not be construed as limited to only the examples set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example examples of the present invention. As used herein, the term “and/or,” includes any and all combinations of one or more of the associated listed items. The phrase “at least one of” has the same meaning as “and/or”.

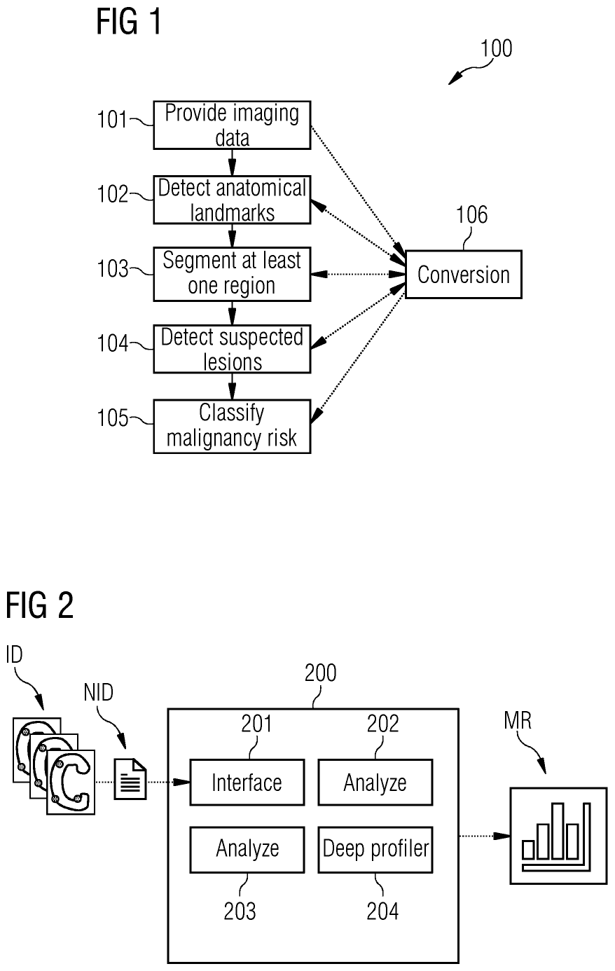

FIG. 1 is a schematic drawing depicting a computer-implemented method 100 for classifying a malignancy risk MR of a kidney.

In a first method act 101, imaging data ID of an anatomy of a subject patient is provided. The imaging data ID includes at least partially a representation of a kidney of the subject patient. The imaging data ID may be based on image capturing via computer tomography (CT) and/or magnet resonance imaging (MRI).

Thus, the provided imaging data ID may include at least partially a 3D illustration of the anatomy of the subject patient. In particular, the 3D illustration is layered, which means that at least two layers can be extracted from the imaging data ID defining at least two different depths of the captured anatomy.

In a second method act 102, a fourth neural network is used to detect anatomical landmarks based on the provided imaging data ID. This act is in particular important since the method may require a precise, automatic detection of anatomical structures, preferably of at least a kidney, to initialize and constrain mathematical models for volumetric organ segmentation. As such, enabling accurate and efficient anatomical landmark detection can support the method 100 for a more effective and streamlined image reading.

Therefore, the fourth neural network may be designed as convolutional neural network for image feature extraction. Furthermore, the fourth neural network may additionally use at least one universal non-linear function approximator.

The network may be parametrized by Θ=[W, b], where W denotes the inter-neural connection weights organized as (multichannel) filter kernels, and b defines the set of neuron bias values.

Convolutional layers may exploit local spatial correlations of image voxels to learn translation-invariant convolutional kernels, which capture discriminative image features. It may be considered a multi-channel signal representation Mₖ in layer k, i.e., a channel-wise concatenation of signal representations M_{k,c} with c∈ [symbol] . One can generate a signal representation in layer k+1 as: M_{k+1,l}=ϕ(Mₖ*w_{k,l}+b_{k,l}), where W_{k,l}∈W may represent a convolutional kernel with the same number of channels as Mₖ, the value b_{k,l}∈b may represent the bias, l denotes the channel index, and * denotes a convolution operation.

The function ϕ may represent the nonlinear activation function, which is applied pointwise. Rectified linear unit (ReLU) activations may be used. The final network layers may be typically fully-connected. In a supervised regression setup, given training data D=[(X_{1,y1}), . . . , (X_{N,YN})] i.e., N independent pairs of volumetric image observations with value assignments, one may define the network response function as R(·;θ), and use Maximum Likelihood Estimation to estimate the optimal network parameters (L denotes the likelihood):

$$\hat{θ} = arg max L ( θ ; D ) = arg min {\sum }_{i = 1}^{N} {( R ( {X}_{i} ; θ ) - {y}_{i} )}^{2}$$

This optimization problem may be solved with stochastic gradient descent (SGD) combined with the backpropagation algorithm to compute the network gradients.

It may be beneficial to reformulate the anatomy detection as a cognitive learning task for an artificial agent. Given a volumetric image I: [symbol] ³→ [symbol] and the location of an anatomical structure of interest \vec{p}_{GT}∈ [symbol] ³ within I, the task may learn a navigation strategy to \vec{p}_{GT} in image space, i.e., a voxel grid of the imaging data ID. In other words, to seek voxel-based navigation trajectories from any arbitrary starting point \vec{p}₀ to \vec{p}ₖ within image I, with the property that ∥\vec{p}ₖ−\vec{p}_{GT}∥ is minimal. With reinforcement learning this problem may be modelled using a Markov Decision Process (MDP) M:=(S, A, T, R, γ) where:
S may represent a finite set of states, sₜ∈S being the state of the agent at time t. To encode the location of the agent in the imaged volumetric space at time t, it may be defined sₜ=I(\vec{p}ₖ), which may denote an axis-aligned box of image intensities extracted from I and centered at the voxel-position \vec{p}ₜ in image space.
A may represent a finite set of actions allowing the agent to interact with the environment defined by I, where aₜ∈A is the action the agent may perform at time t. A discrete voxel-wise navigation model may be used allowing the agent to move from any voxel position \vec{p}ₜ to an adjacent voxel position \vec{p}ₜ₊₁ in image space.
T:S×A×S→[0;1] may be a stochastic transition function, where T_{s,a}^{s′} may describe the probability of arriving in state s′, after performing action a in state s.
R:S×A×S→ [symbol] may be a scalar reward function, which drives the behavior of the agent, where R_{s,a}^{s′}∈ [symbol] may denote the expected reward after a state transition. For a state transition s→s′ at time t from \vec{p}ₜ→\vec{p}ₜ₊₁, we define R_{s,a}^{s′}=∥\vec{p}ₜ−\vec{p}_{GT}∥₂²−∥\vec{p}ₜ₊₁−\vec{p}_{GT}∥₂². This may represent a distance-based feedback, which is positive if the agent gets closer to the target structure and negative otherwise. γ may be the discount factor controlling the importance of future versus immediate rewards.

Furthermore, an optimal action-value function Q*( . . . , . . . ) may be defined that encodes the maximum expected future discounted reward when starting in state s, performing action a, and acting optimally thereafter:
Q*(s,a)=maxE[Rₜ|sₜ=s,aₜ=a,π], where π may be an action policy, in other words a probability distribution over actions in any given state. An important relation satisfied by the optimal action-value function Q* may be the Bellman optimality equation, which represents following recursive formulation:
Q*(s,a)=ΣT_{s,a}^{s′}(R_{s,a}^{s′}+γmaxQ*(s′,a′))=E_{s′}(r+γmaxQ*(s′,a′)), where s′ defines a possible state visited after s, a′ the corresponding action and r=R_{s,a}^{s′} represents a compact notation for the current, immediate reward. In one example, this approach may be amended by deploying a deep Q-network (DQN) which is used as a non-linear approximator for the optimal action-value function. Accordingly, a deep Q-network can be trained in an RL setup using an iterative approach to minimize the mean squared error based on the Bellman optimality equation. At any training-iteration i, it may be possible to approximate an optimal expected target value for the action-value function using a set of reference parameters based on a previous training iteration i′<i.

Learning the action-value function Q* may enable the agent to effectively search for objects in the image, as opposed to scanning the volumetric space exhaustively. This learning process may be based on an adequate exploration of the environment, which we ensure through an off-policy ∈-greedy approach.

The variable ∈ε[0,1] controls the randomness in the exploration. This means that during training, actions are selected either uniformly at random with probability ∈, or deterministically using the current policy with probability 1−∈. Another important strategy to ensure the training stability may be the decorrelation of the training samples using the concept of experience replay. During training, the agent maintains an active memory of episodic trajectories M=[T1; T2; . . . ], which is constantly expanded and uniformly sampled to estimate the learning gradient.

To further accelerate the training, it may be possible to use an adaptive episode length. By gradually reducing the episode length during training using linear decay, the space exploration by sampling increasing numbers of trajectories that are stored in the active memory may be improved.

In a third method act 103, a first neural network is used to segment at least one region of the kidney representation based on the imaging data ID. Therefore, the first neural network may be designed as convolutional encoder-decoder architecture. Additionally, the first neural network may be designed as multi-level feature concatenation and deep supervision architecture. Compared to non-learning-based approaches like statistical distribution of the intensity, including atlas-based, active shape model (ASM-) based, level-set based or graph-cut-based methods, learning-based approaches may be more beneficial for better segmentation.

Fully convolutional networks (FCN) with deep supervision may be used, which can perform end-to-end learning and inference. The output of FCN may be refined with a fully connected conditional random field (CRF) approach. Furthermore, cascaded FCNs followed by CRF refinement may be applied.

However, also Generative Adversarial Networks (GAN) may be a powerful framework for this task. The GAN may include at least a generator and a discriminator. The generator tries to produce the output that is close to the real samples, while the discriminator attempts to distinguish between real and generated samples.

An advanced approach may be an adversarial image-to-image network (DI2IN-AN), wherein a deep image-to-image network (DI2IN) may serve as a generator to produce a liver segmentation. It may employ a convolutional encoder-decoder architecture combined with multi-level feature concatenation and deep supervision. The network may try to optimize a conventional multi-class cross-entropy loss together with an adversarial term that aims to distinguish between the output of DI2IN and ground truth.

Ideally, the discriminator pushes the generator's output towards the distribution of ground truth, so that it may have the potential to enhance generator's performance by refining its output. Since the discriminator is usually a CNN that takes the joint configuration of many input variables, it may embed the higher-order potentials into the network (the geometric difference between prediction and ground truth is represented by the trainable network model instead of heuristic hints). The proposed implementation also achieves higher computing efficiency since the discriminator does not need to be executed at inference.

DI2IN may take the 3D imaging data ID as input and outputs the probability maps that indicate how likely voxels belongs to the liver region. At least one block, in particular all blocks in DI2IN include 3D convolutional and bilinear upscaling layers.

In the encoder part of DI2IN, only the convolution layers are used in all blocks. In order to increase the receptive field of neurons and lower the GPU memory consumption, stride may be set as 2 at some layers and the size of feature maps may be reduced. Moreover, larger receptive field may cover more contextual information and help to preserve liver shape information in the prediction. The decoder of DI2IN may include convolutional and bilinear upscaling layers.

To enable end-to-end prediction and training, the upscaling layers may be implemented as bilinear interpolation to enlarge the activation maps. All convolutional kernels may be of 3×3×3. The upscaling factor in a decoder may be 2 for x; y; z dimension. The leaky rectified linear unit (Leaky ReLU) and batch normalization may be adopted in all convolutional layers for proper gradient back-propagation.

In order to further improve the performance of DI2IN, several mainstream technologies may be adopted. First, a feature layer concatenation may be used in DI2IN. Fast bridges may be built directly from the encoder layers to the decoder layers. The bridges may pass the information from the encoder forward and then may concatenate it with the decoder feature layers. The combined feature may be used as the input for the next convolution layer. Following the acts above to explicitly combine advanced and low-level features, DI2IN may benefit from local and global contextual information.

In a fourth method act 104, a second neural network is used to detect one or more suspected lesions of the segmented kidney representation. Therefore, the third neural network may be configured to classify the malignancy risk MR based on imaging data ID and non-imaging data NID, wherein the non-imaging data NID includes at least histopathologic data.

The second neural network may be configured at least partly as a fully convolutional one-stage object detection (FCOS). This is preferably performed in a per-pixel prediction fashion, analogue to semantic segmentation. The FCOS may be anchor box free and/or proposal free. Thus, the FCOS may avoid any complicated computation related to anchor boxes such as calculating overlapping during training. Additionally, FCOS may avoid hyper-parameters related to anchor boxes, which may be sensitive to a final detection performance.

An example of a fully convolutional one-stage object decoder may be defined as follows: Let Fᵢ∈ [symbol] ^{H×W×C} be the feature maps at layer I of a backbone CNN and s be the total stride until the layer. The ground-truth bounding boxes for the imaging data ID may be defined as {Bᵢ}, where Bᵢ=(x₀⁽ⁱ⁾, y₀⁽ⁱ⁾, x₁⁽ⁱ⁾, y₁⁽ⁱ⁾, c⁽ⁱ⁾)∈ [symbol] ⁴×{1,2 . . . C}. (x₀⁽ⁱ⁾, y₀⁽ⁱ⁾) and (x₁⁽ⁱ⁾, y₁⁽ⁱ⁾) may denote the coordinates of the left-top and right-bottom corners of the bounding box. c⁽ⁱ⁾ may be the class that the object in the bounding box belongs to. C may be the number of classes. For each location (x, y) on the feature map Fᵢ, one may map it back onto the input image as

$$( [ \frac{s}{2} ] + xs , [ \frac{s}{2} ] + ys ) ,$$

which is near the center of the receptive field of the location (x, y). The target bounding box may be directly regressed at the location.

Location (x, y) may be considered as a positive sample if it falls into any ground-truth box and the class label c* of the location may be the class label of the ground-truth box. Otherwise, it may be a negative sample and c*=0 (background class). Furthermore, there may be a 4D real vector t*=(l*, t*, r*, b*) being the regression targets for the location. l*, t*, r* and b* may be distances from the location to the four sides of the bounding box. If a location falls into multiple bounding boxes, it may be considered as an ambiguous sample. The bounding box with minimal area may be chosen as its regression target.

If location (x, y) is associated to a bounding box Bᵢ, the training regression targets for the location may be formulated as, l*=x−x₀⁽ⁱ⁾, t*=y−y₀⁽ⁱ⁾, r*=x₁⁽ⁱ⁾−x and b*=y₁⁽ⁱ⁾−y. The FCOS may leverage as many foreground samples as possible to train the regressor.

Corresponding to the training targets, the final layer of the networks may predict an 80D vector p of classification labels and a 4D vector t=(l, t, r, b) bounding box coordinates. In one example, C binary classifiers may be trained. In one example, at least four convolutional layers may be added after the feature maps of the backbone networks respectively for classification and regression branches. Since regression targets may be positive, exp(x) may be employed to map any real number to (0,∞) on the top of the regression branch.

A training loss function may be defined as follows:

$$L ( \{ {p}_{x , y} \} , \{ {t}_{x , y} \} ) = \frac{1}{{N}_{pos}} {\sum }_{x , y} {L}_{cls} ( {p}_{x , y} , {c}_{x , y}^{*} ) + \frac{λ}{{N}_{pos}} {\sum }_{x , y} {1}_{\{ {c}_{x , y}^{*} > 0 \}} {L}_{reg} ( {t}_{x , y} , {t}_{x , y}^{*} ) ,$$

where L_{cls} may be focal loss and Lreg may be the IOU loss. Nₚₒₛ may denote the number of positive samples and λ being in 1 may be the balance weight for L_{reg}. The summation may be calculated over all locations on the feature maps Fᵢ. 1_{{c}_{_{x,y}}_{^{*}}_{>0}} may be the indicator function, being 1 if cᵢ^{*}>0 and 0 otherwise.

Giving the imaging data ID, they may be forwarded through the network and may obtain the classification scores p_{x,y} and the regression prediction t_{x,y} for each location on the feature maps Fᵢ. In one example, the required IOU scores for positive anchor boxes may be lowered.

In one example, a single-layer branch may be added additionally, in parallel with a classification branch, in order to predict a “centerness” of a location. The centerness may depict the normalized distance from the location to the center of the object that the location is responsible for. Given the regression targets 1*, t*, r* and b* for a location the centerness target may be defined as

$${centerness}^{*} = \sqrt{\frac{min ( {l}^{*} , {r}^{*} )}{max ( {l}^{*} , {r}^{*} )} \times \frac{min ( {t}^{*} , {b}^{*} )}{max ( {t}^{*} , {b}^{*} )}} .$$

The centerness may range from 0 to 1 and may be trained with binary cross entropy (BCE) loss. The final score may be computed by multiplying the predicted centerness with the corresponding classification score.

In a fifth method act 105, the detected suspected lesion may be classified with a malignancy risk MR using a third neural network, wherein the third neural network is a deep profiler. Therefore, the deep profiler may include an encoder for extracting imaging features. Furthermore, the encoder may be designed as a convolutional neural network (CNN), in particular a three-dimensional CNN. Additionally, the deep profiler may include a decoder for estimating at least one malignancy risk MR indicator and/or a task-specific network for generating at least one image signature for classifying at least one malignancy risk MR.

In one example, the imaging data ID may be quantified by intensity, geometry, texture and/or wavelet features. The geometry features may quantify the 2D or 3D shape characteristics of the kidney, the texture features may describe spatial distribution of voxel or pixel intensities, thereby quantifying a heterogeneity. Any intensity and texture features may be computed after applying wavelet transformations to the imaging data ID.

To find voxels of the imaging data ID that contribute the most toward the prediction, the derivative of the final partial likelihood loss with respect to the imaging data ID may be taken and evaluated.

In one example, the imaging data ID and/or non-imaging data NID may include context data relating to data recording information. In addition, the context data may include at least one out of following: data recording device information, contrast configuration information, brightness configuration information, recording direction information, total recording time information, projection type information (e.g. CT: average intensity projection (AIP), maximum intensity projection (MIP) or Minimum intensity projection MinIP)), date and/or time of recording information.

In one example, the non-imaging data NID of the subject patient may include at least one out of following: age, weight, size, health condition information, gender, nutritional practice.

A sixth method act 106, may convert the imaging data ID from at least a partially 3D illustration of the anatomy of the subject patient to a 2D illustration of the anatomy of the subject patient. This method act may be performed at any time due to, e.g., memory or computational limitations.

FIG. 2 is a schematic drawing depicting a system 200 for classifying a malignancy risk MR scoring of a kidney.

The system 200 includes an interface 201 configured to provide imaging data ID of an anatomy of a subject patient, wherein the imaging data ID includes at least partially a representation of a kidney of the subject patient. Therefore, imaging data ID will be forwarded, in particular together with non-imaging data, to the interface.

A first analyzing unit 202 (processor with analyzing instructions) of the system 200 is configured to use a first neural network to segment at least one region of the kidney representation which is based on the imaging data. A second analyzing unit 203 (processor with analyzing instructions) is also part of the system 200, wherein the second analyzing unit 203 is configured to use a second neural network to detect one or more suspected lesions of the segmented kidney representation. The system includes additionally a deep profiler 204 (processor with deep profiler instructions) which is configured to classify the detected suspected lesion with a malignancy risk MR.

In one example, the deep profiler 204 is configured to classify the malignancy risk MR based on imaging data ID and non-imaging data NID, wherein the non-imaging data NID includes at least histopathologic data.

Whenever a malignancy risk MR score has been determined by the system 200, the value may be stored in the system 200 (storage in memory) or in a cloud-based memory storage system. In one example, at least parts of the system 200 may be deployed in a cloud-based system, which means that they do not have to be physically integrated in a box.

The system 200 may be configured that each input data and/or output data can be forwarded to each of its units, namely its interface 201, its first analyzing unit 202, its second analyzing unit 203 and/or its deep profiler 204. It is further denoted that the neural networks of the specific units may be designed analogue to the neural networks disclosed above relating to FIG. 1.

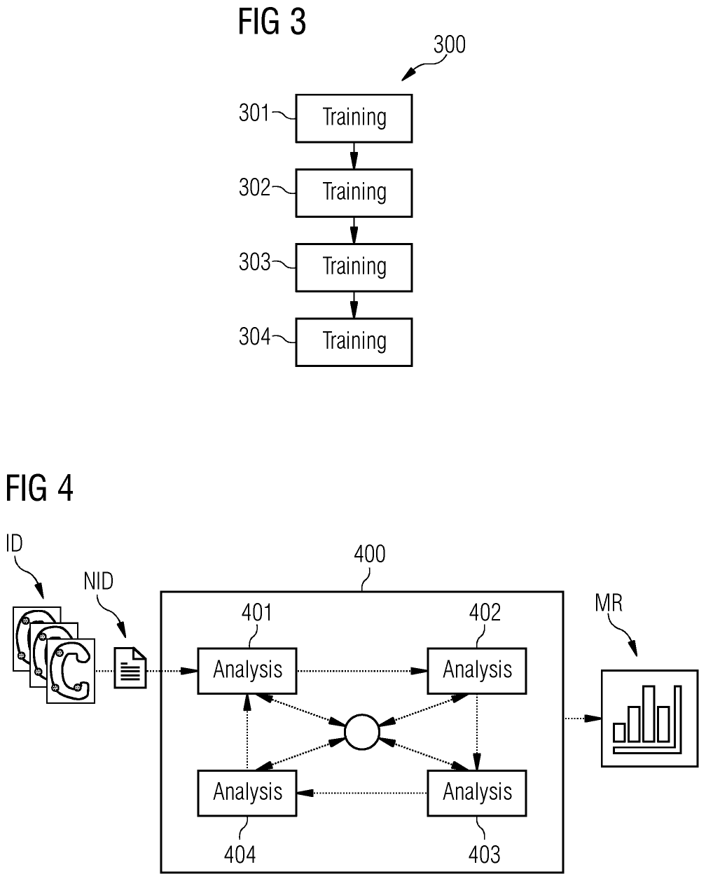

FIG. 3 is a schematic drawing depicting a computer-implemented method 300 for training a machine learning algorithm to classify a malignancy risk MR of a kidney.

Therefore, the method 300 includes a first act 301 of training a first neural network with first training data including imaging data ID of an anatomy of at least one subject patient, wherein the imaging data ID includes at least partially a representation of one or more kidneys. An adversarial network may be utilized for training of the first neural network to discriminate the output from ground truth.

In a second method act 302, a second neural network is trained with second training data including one or more detected lesions of one or more segmented kidney representations.

In a third method act 303, a third neural network is trained with third training data of one or more lesions classified with a malignancy risk MR. The third training data are preferably based on imaging data ID and non-imaging data NID, wherein the non-imaging data NID includes at least histopathologic data. Furthermore, at least one ground-truth label of the deep profiler may be determined based on histopathologic data.

In a fourth method act 304, a fourth neural network is trained using deep reinforcement learning based on fourth training data related to detected anatomical landmarks, in particular landmarks relating to one or more kidney representations.

In one example at least two different training data fragments of the first, second third or fourth training data may be based on the same subject patient. Furthermore, the two different training data fragments may be indicated as training data of the same subject patient. Additionally, the non-imaging data NID may include at least one indicator which is configured to serve for patient follow-up diagnosis.

FIG. 4 is a schematic drawing depicting a system 400 for training a machine learning algorithm to classify a malignancy risk MR of a kidney.

Therefore, the system 400 includes a first analysis unit 401 (processor with analyzing instructions) which is configured to train a first neural network with first training data including imaging data ID of an anatomy of at least one subject patient, wherein the imaging data ID includes at least partially a representation of one or more kidneys.

A second analysis unit 401 (processor with analyzing instructions) of the system 400 is configured to train a second neural network with second training data including one or more detected lesions of one or more segmented kidney representations. The system 400 further includes a third analysis unit 403 (processor with analyzing instructions) configured to train a third neural network with third training data of one or more lesions classified with a malignancy risk MR.

The system may additionally include a fourth analysis unit 404 (processor with analyzing instructions) configured to train a fourth neural network with fourth training data related to detected anatomical landmarks, in particular landmarks relating to one or more kidney representations.

The system 400 may be configured that each input data and/or output data can be forwarded to each of its units, namely its first analysis unit 401, its second analysis unit 402, its third analysis unit 403 and/or its fourth analysis unit 404. It is further denoted that the neural networks of the specific units may be designed analogue to the neural networks disclosed above relating to FIG. 3.

According to one embodiment the following the following clause is provided:
Clause 1: A computer-implemented method (100) for classifying a malignancy risk (MR) of a kidney, in particular a human kidney, comprising following acts:
   providing (101) imaging data (ID) of an anatomy of a subject patient, wherein the imaging data (ID) comprises at least partially a representation of a kidney of the subject patient;
   using (102) a first neural network to segment at least one region of the kidney representation which is based on the imaging data (ID);
   using (103) a second neural network to detect one or more suspected lesions of the segmented kidney representation; and
   classifying (104) the detected suspected lesion with a malignancy risk (MR) using a third neural network,
   wherein the third neural network is a deep profiler.
2. The method of clause 1, wherein the imaging data (ID) and/or non-imaging data (NID) comprises context data relating to data recording information.
3. The method of clause 1 or 2, wherein the context data comprises at least one out of following:
   data recording device information, contrast configuration information, brightness configuration information, recording direction information, total recording time information, projection type information, date and/or time of recording information.
4. The method of any of clauses 1 to 3, wherein the non-imaging data (NID) of the subject patient comprises at least one out of following:
   age, weight, size, health condition information, gender, nutritional practice.
According to a 5^{th} clause, the following is provided:
   A computer-implemented method (300) for training a machine learning algorithm to classify a malignancy risk (MR) of a kidney, in particular a human kidney, comprising following acts:
   training (301) a first neural network with first training data including imaging data (ID) of an anatomy of at least one subject patient, wherein the imaging data (ID) comprises at least partially a representation of one or more kidneys;
   training (302) a second neural network with second training data including one or more detected lesions of one or more segmented kidney representations; and training (303) a third neural network with third training data of one or more lesions classified with a malignancy risk.
6. The method of clause 5, wherein the third training data are based on imaging data (ID) and non-imaging data (NID), wherein the non-imaging data (NID) comprises at least histopathologic data.
7. The method of clause 5 or 6, wherein at least one ground-truth label of the deep profiler is determined based on histopathologic data.
8. The method of any of clauses 5 to 7, comprising following additional act:
   training (304) a fourth neural network using deep reinforcement learning based on fourth training data related to detected anatomical landmarks, in particular landmarks relating to one or more kidney representations.
9. The method of any of clauses 5 to 8, wherein an adversarial network is utilized for training of the first neural network to discriminate the output from ground truth.
10. The method of any of clauses 5 to 10, wherein at least two different training data fragments are based on the same subject patient.
11. The method of clause 10, wherein the two different training data fragments are indicated as training data of the same subject patient.
12. The method of any of clauses 5 to 11, wherein the non-imaging data (NID) comprise at least one indicator which is configured to serve for patient follow-up diagnosis.

Although the present invention has been described in detail with reference to the preferred example, the present invention is not limited by the disclosed examples from which the skilled person is able to derive other variations without departing from the scope of the invention. Example examples being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for classifying a malignancy risk of a kidney, the method comprising:
providing imaging data of an anatomy of a subject patient, wherein the imaging data comprises at least partially a representation of the kidney of the subject patient;
segmenting using a first neural network at least one region of the kidney representation based on the imaging data;
detecting using a second neural network one or more suspected lesions of the segmented kidney representation; and
classifying the detected suspected lesion with the malignancy risk using a third neural network,
wherein the third neural network is a deep profiler.

2. The method of claim 1, wherein the third neural network classifies the malignancy risk based on imaging data and non-imaging data, wherein the non-imaging data comprises at least histopathologic data.

3. The method of claim 1, wherein classifying comprises classifying using the deep profiler, the deep profiler comprising an encoder for extracting imaging features.

4. The method of claim 3, wherein the encoder is a convolutional neural network.

5. The method of claim 3, wherein the deep profiler comprises a decoder, and wherein classifying comprises estimating at least one malignancy risk indicator by the decoder.

6. The method of claim 1, wherein the deep profiler comprises a task-specific network, and wherein classifying comprises generating at least one image signature for classifying the malignancy risk using the task-specific network.

7. The method of claim 1, further comprising:
detecting anatomical landmarks using a fourth neural network based on the provided imaging data.

8. The method of claim 7 wherein the fourth neural network is a convolutional neural network using at least one universal non-linear function approximator, and wherein classifying comprises extracting an image feature by the convolutional neural network.

9. The method of claim 1, wherein the first neural network is a convolutional encoder-decoder architecture or a multi-level feature concatenation and deep supervision architecture.

10. The method claim 1, wherein detecting the one or more suspected lesions comprises detecting based on a fully convolutional one-stage object detection of the second neural network.

11. The method of claim 1, wherein providing the imaging data comprises at least one of the following: providing based on computer tomography and/or magnet resonance imaging, and/or
providing at least partially a 3D illustration of the anatomy of the subject patient.

12. The method of claim 1, further comprising:
converting the imaging data from at least a partially 3D illustration of the anatomy of the subject patient to a 2D illustration of the anatomy of the subject patient.

13. A system for classifying a malignancy risk scoring of a kidney, the system comprising:
an interface configured to provide imaging data of an anatomy of a subject patient, wherein the imaging data comprises at least partially a representation of a kidney of the subject patient;
a processor configured to use a first neural network to segment at least one region of the kidney representation which is based on the imaging data, configured to use a second neural network to detect one or more suspected lesions of the segmented kidney representation, and configured to implement a deep profiler to classify the detected suspected lesion with a malignancy risk.

14. The system of claim 13, wherein the deep profiler is configured to classify the malignancy risk based on imaging data and non-imaging data, wherein the non-imaging data comprises at least histopathologic data.

15. The system of claim 13, wherein the deep profiler comprises an encoder to extract imaging features.

16. The system of claim 13, wherein the deep profiler comprises a decoder configured to estimate at least one malignancy risk indicator.

17. The system of claim 13, wherein the deep profiler comprises a task-specific network configured to generate at least one image signature for classification of the malignancy risk using the task-specific network.

18. A method for training a machine learning algorithm to classify a malignancy risk of a kidney, the method comprising:
training a first neural network with first training data including imaging data of an anatomy of at least one subject patient, wherein the imaging data comprises at least partially a representation of one or more kidneys;
training a second neural network with second training data including one or more detected lesions of one or more segmented kidney representations; and
training a third neural network with third training data of one or more lesions classified with a malignancy risk.

19. The method of claim 18, wherein training the third neural network comprises training a deep profiler.
